(19) United States
(12) Patent Application Publication
(10) Pub. No.: US 2021/0348099 A1
(43) Pub. Date: Nov. 11, 2021

(54) DEVICES FOR INCUBATING AND WASHING BIOLOGICAL SAMPLES AND METHODS USING THEREOF

(71) Applicants: Rongrong Wu, El Sobrante, CA (US); Guofu Wang, Wuhan (CN); Suheng Liu, Wuhan (CN)

(72) Inventors: Rongrong Wu, El Sobrante, CA (US); Guofu Wang, Wuhan (CN); Suheng Liu, Wuhan (CN)

(21) Appl. No.: 16/869,585

(22) Filed: May 7, 2020

(51) Int. Cl. C12M 3/00; C12M 1/00; C12M 1/36

(52) U.S. Cl. CPC C12M 23/48; C12M 23/50; C12M 41/48; C12M 41/14

(57) ABSTRACT

The present disclosure provides devices, machines and methods for incubating or washing a biological sample. This disclosure provides a device comprising a motor and a control for the motor, a supporting frame comprising a receptacle, an assembly comprising a rotatable liquid storage container, a washing cassette, and a liquid collection and recovery tank. The disclosure also provides methods using the device.

### BACKGROUND

Biological experiments require processing membranes or gels containing biological sample. Biological samples may include deoxyribonucleic acid (DNA), ribonucleic acid (RNA), sugar, lipid, protein, etc. The processing methods may be nucleic acid hybridization, detection using antibodies, and staining, etc.

Take Western blot or protein immunoblot as an example, the traditional Western blot methods have been around for more than 40 years. The process includes incubating the protein-immobilized membrane with antibodies and then washing the membrane. In the lab, manual labor is required to perform Western blot experiments. There are automation alternatives on the market. For example, devices that can automatically incubate and wash the samples. Many such machines use a shaker to shake a container having the membrane in a solution horizontally, and use a liquid pump to supply liquid/solution to the container.

### SUMMARY

The following embodiments and aspects thereof are described and illustrated in conjunction with devices, systems and methods which are meant to be exemplary and illustrative, not limiting in scope.

Because of the complexity, high price, and high antibody consumption required when using commercial machine to run Western blot experiments, there is a need to design better, cheaper and simpler machine to accomplish the incubation and washing steps of Western blot. The present disclosure discloses a motor-controlled mechanical device to complete the whole process of incubation and washing of the Western blot membrane. In addition, the cost of is very competitive compared to current machines on the market. The operation of the device is simple, and the consumption of the antibodies is small. In some embodiments, the antibody can be recovered. In some embodiments, there is no or little cross contamination between different antibodies during the Western blot experiment.

The present disclosure provides a device for incubating and washing biological samples, including a V-shaped support frame and a liquid collection and recovery tanks arranged inside the V-shaped support frame.

In one aspect, the present disclosure provides a device for incubating or washing a biological sample, the device comprising: (a) a support frame comprising an upper part and a lower part; (b) a rotatable liquid storage container assembly disposed at the upper part of the support frame, wherein the rotatable liquid storage container is configured to store and transfer a liquid; and (c) a sample holder assembly disposed at the lower part of the support frame, wherein the sample holder comprises: (i) a sample holder configured to receive a biological sample; and (ii) an upper basin disposed between the sample holder and the rotatable liquid storage container; wherein the upper basin is engaged with both the sample holder and the rotatable liquid storage container assembly.

In some embodiments, the rotatable liquid storage container assembly comprises a first liquid storage container and a first drive gear, wherein the first liquid storage container comprises a first chamber comprising (i) an open-ended top, (ii) two vertical sides opposing each other, and (iii) a bottom side comprising matching driven gear to mesh with the first drive gear, thereby the first drive gear drives the first liquid storage container. In some embodiments, the each of the two vertical sides of the first chamber comprises an opening configured for a rod to pass through, wherein the first liquid storage container further comprises an inserted container disposed in the first chamber, wherein the inserted container comprises a cavity configured for the rod to pass through, and wherein the rod secures the first liquid storage container to the support frame. In some embodiments, the first chamber further comprises a pouring lip on the open-ended top. In some embodiments, the rotatable liquid storage container assembly further comprises a second liquid storage container and a second drive gear, wherein the second liquid storage container comprises a second chamber, wherein the second drive gear drives the second liquid storage container. In some embodiments, the first drive gear is configured not to drive the second liquid storage container, and wherein the second drive gear is configured not to drive the first liquid storage container. In some embodiments, the first drive gear and the second drive gear are configured not to share a common coordinate on any of the X-axis, Y-axis, and Z-axis of a 3-dimensoional Cartesian coordinate system. In some embodiments, the further comprising (d) a liquid collection and recovery tank disposed at the lower part of the support frame and below the sample holder assembly.

In some embodiments, the device further comprises a volume occupier disposed within the sample holder. In some embodiments, the device further comprises a sample holder drive gear configured to operate the sample holder. In some embodiments, the sample holder further comprises: a washing cylinder disposed within the sample holder, wherein the washing cylinder is cylindrical with two open ends, and wherein the washing cylinder comprises a plurality of slots. In some embodiments, the sample holder further comprises: a curved surface at the lower part of the sample holder; wherein the curved surface comprises a plurality of poking teeth configured to poke the washing cylinder by protruding through the plurality of slots. In some embodiments, a system is provided to comprise a plurality of the devices. In some embodiments, the system further comprises a processor to control the plurality of the device. In some embodiments, the processor is configured to control the operation of rotatable liquid storage container assembly and the sample holder.

Further areas of applicability of the present disclosure will become apparent from the detailed description provided hereinafter. It should be understood that the detailed description and specific examples, while indicating various embodiments, are intended for purposes of illustration only and are not intended to limit the scope of the disclosure.

### BRIEF DESCRIPTION OF THE DRAWINGS

The object and features of the present disclosure can be more fully understood and better appreciated with reference to the attached drawings, which are schematic representations only and not necessarily drawn to scale, wherein:

FIG. 1 shows an illustration of an example device 100 for incubating and washing a biological sample of the present disclosure.

FIG. 2 is an exploded view of an example of a rotatable liquid storage container assembly 200.

FIG. 3 shows an illustration of an example of another rotatable liquid storage container assembly 300.

FIG. 4 shows an illustration of an example of a sample holder assembly 400.

FIG. 5 shows an illustration of an upper basin 500 of the sample holder assembly shown in the FIG. 4.

FIG. 6 shows an illustration of a partial view of an example sample holder assembly 600.

FIG. 7 shows an illustration of a partial view of another example sample holder assembly 700 and an example support frame.

FIG. 8 shows an exploded, partial view of an example sample holder assembly 800.

FIG. 9 is shows a section view of an example sample holder assembly 900.

FIG. 10 shows a perspective view of an example sample holder 1000.

FIG. 11 provides another perspective view of an example sample holder 1100.

FIG. 12 shows a cartoon of the process 1200 to pour a liquid from a rotatable liquid storage container to an upper basin.

FIG. 13 provides a cartoon of the process 1300 to pour a liquid from a sample holder to a liquid collection and recovery tank.

FIG. 14 depicts a partial view of a sample device 1400 of the present disclosure.

FIG. 15 shows an example system 1500 comprising multiple copies of a device of the present disclosure.

FIG. 16 depicts another example system 1600 comprising multiple copies of a device of the present disclosure.

### DETAILED DESCRIPTION

The present disclosure relates to an improved apparatus and methods for processing biological samples using automation. Various modifications to the disclosed embodiment will be readily apparent to those skilled in the art and the principles herein may be applied to other embodiments and combination thereof. Although various components are discussed in the context of a particular initial design, it should be understood that the various elements can be altered and even replaced or omitted to permit other designs and functionality. Thus, the present disclosure is not intended to be limited to the embodiments shown but is to be accorded the widest scope consistent with the principles and features described herein. As used herein, the “present disclosure” or “present application” refers to any one of the embodiments of the disclosure described herein, and any equivalents thereof. Furthermore, reference to various feature(s) of the “present disclosure” or “present application” throughout this document does not mean that all claimed embodiments or methods must include the referenced feature(s).

### NUMERALS

100 Device
1 rotatable liquid storage container assembly
2 sample holder assembly
3 liquid collection and recovery tank
4 support frame
200 rotatable liquid storage container assembly
11 chamber
12 opening
13 drive gear
14 inserted container
15 pouring lip
300 rotatable liquid storage container assembly
400 sample holder assembly
21 upper basin
22 sample holder
211 liquid collection basin
213 liquid conduit
214 liquid outlet
221 washing cylinder
222 cylinder drive gear
223 lower frame
500 upper basin
212 cylinder block
215 V-shaped block
600 sample holder assembly
2231 cylinder driven gear
2232 curved surface
2233 first lower frame pouring lip
2234 orifice
2237 second lower frame pouring lip
700 sample holder assembly
41 V-shaped protrusion
800 sample holder assembly
2151 retaining slot
2152 retaining spring clip
2235 groove
2236 second poking tooth
900 sample holder assembly
216 first poking tooth
1000 sample holder
224 sample holder drive gear
1100 sample holder
5 Western blotting membrane
6 volume occupier
1200 process to pour a liquid from a rotatable liquid storage container to an upper basin
1300 Process to pour a liquid from a simple holder to a liquid collection and recovery tank
31 antibody collection and recovery tank
32 waste collection tank
1400 device
1500 system comprising multiple copies of a device
1600 system comprising multiple copies of a device

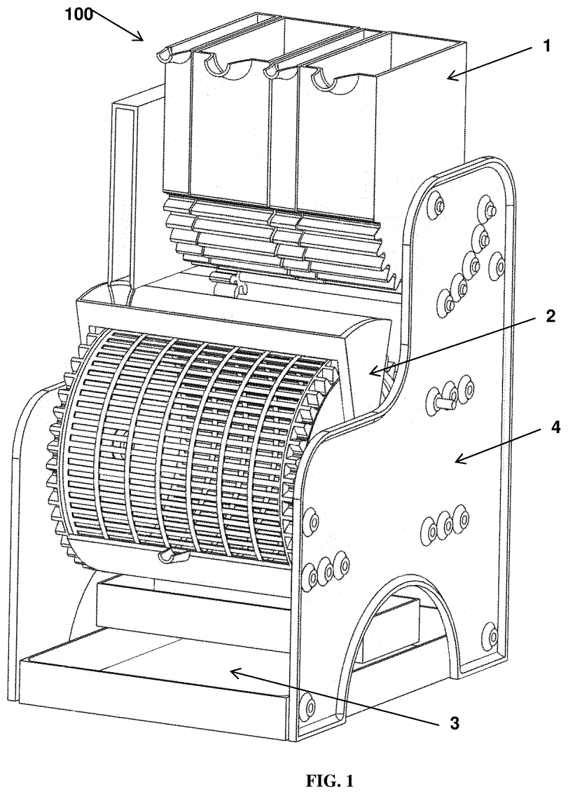

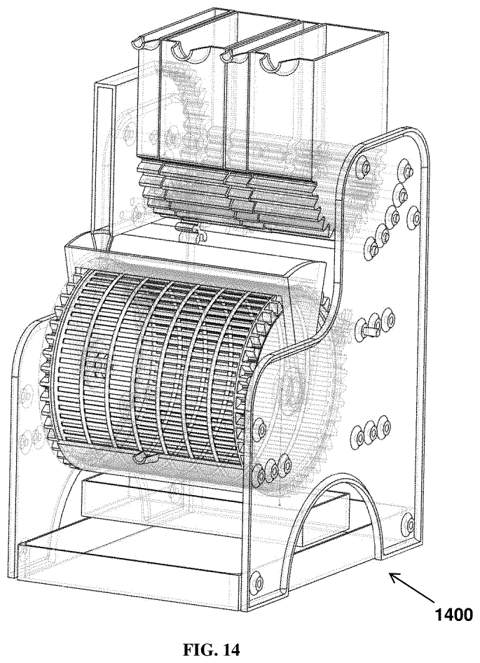

To appreciate the features and advantages of preferred apparatuses and methods in accordance with the present disclosure, the reader is referred to the appended FIGS. 1-21 in conjunction with the following discussion. It is to be understood that the drawings are diagrammatic and schematic representations only and are neither limiting of the scope of the present disclosure nor necessarily drawn to scale. Unless stated otherwise, the same numeral refers to the same element in the specification and drawings of the present disclosure.

### I. Processing Device

In a first aspect, a device is disclosed for incubating or washing a biological sample. As shown in FIG. 1, the device 100 comprises: (a) a rotatable liquid storage container assembly 1; (b) a sample holder assembly 2, (c) a liquid collection and recovery tank 3; and a support frame 4. Arranged from the top to the bottom of the support frame 4 are: the rotatable liquid storage container assembly 1; the sample holder assembly 2, and the liquid collection and recovery tank 3. The device 100 also comprises a motor and a controller for the motor (not shown).

In some embodiments, the rotatable liquid storage container assembly 1 is disposed at the upper part of the support frame and is configured to store and transfer a liquid. As shown below, the rotatable liquid storage container assembly 1 comprises a container that comprises an open-ended top and is configured to pour liquid when powered by an external motor (e.g., a motor-driven gear). In some embodiments, the sample holder assembly 2 at the lower part of the support frame comprises a sample holder 22 and an upper basin 21 which transfers liquid from the rotatable liquid storage container assembly 1 into the sample holder 22. In some embodiments, the liquid collection and recovery tank 3 is disposed at the lower part of the support frame and under the sample holder assembly 2. The liquid collection and recovery tank 3 comprises an antibody collection and recovery tank 31 and a waste collection tank 32, both of which can collect liquids disposed from the sample holder 22.

The biological sample to be processed by the disclosed device can be a membrane-shaped biological sample such as a Western blot membrane or a polyacrylamide gel.

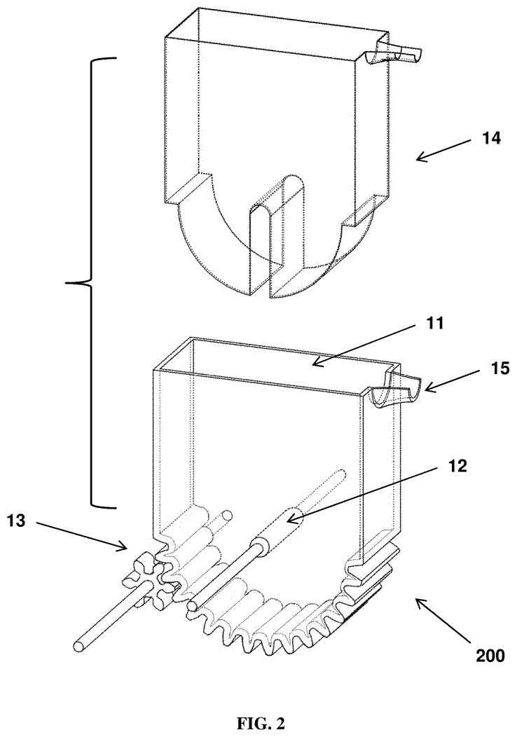

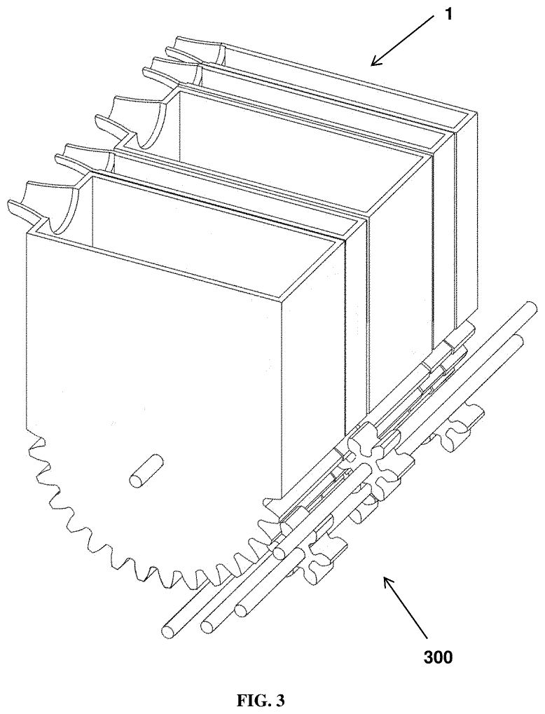

As shown in FIGS. 2 and 3, the device for incubating or washing a biological sample comprises a rotatable liquid storage container assembly 1. FIG. 3 shows a rotatable liquid storage container assembly 1 comprising a plurality of individual rotatable liquid storage 300. FIG. 2 shows an exploded view of an individual rotatable liquid storage 200, which comprises a chamber 11 and an inserted container 14 that can fit into and engage with the chamber 11. The chamber 11 comprises a body, an open upper end on the body for storing/pouring liquid, a pouring lip 15 on the open upper end, a shaft opening 12 crossing the body of the chamber 11 for a shaft to pass through, and a drive gear 13 driven by an external driving device, such as a motor. The inserted container 14 can be prefilled with a liquid before use and have the accommodating shape to allow the shaft to pass though as well. The bottom part of the chamber 11 can take an arc shape and have a plurality of gear teeth to mesh with the drive gear 13, such that the external driving device can rotate the individual rotatable liquid storage 200 such that the liquid stored inside the inserted container 14 can flow out via the pouring lip 15.

The shapes of the vertical sides of the individual rotatable liquid storage container are the same. But each rotatable liquid storage container can have different thicknesses and volume. The drive gears 13 for each rotatable liquid storage container can be staggered such that each drive gear can drive a corresponding rotatable liquid storage container, without interfering with the movement or control of another rotatable liquid storage container. Each individual rotatable liquid storage container can be controlled to pour out the liquid it contains

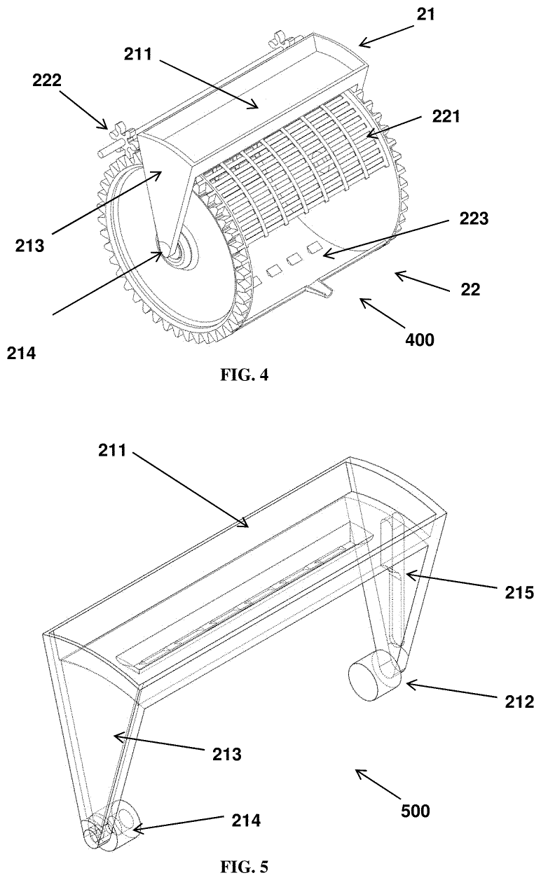

As shown in FIGS. 4, 5 and 7, the upper end of upper basin 21 is a liquid collection basin 211. The liquid collection basin 211 comprises two vertical V-shaped stands. One V-shaped stand is a liquid conduit 213 connected to the liquid collection and recovery tank 3. The lower end of the liquid conduit 213 communicates with the liquid collection tank. The lower end liquid conduit 213 a liquid outlet 214 in fluid communication with the sample holder 22. The other V-shaped stand is V-shaped block 215. The lower part of the V-shaped block 215 comprises a cylinder block 212 configured to insert into and engage with upper basin 21. Each of the inner surfaces of the two V-shaped stands comprises a V-shaped protrusion 41 such that the liquid collection basin and the V-shaped block can insert into one of them.

As shown in FIGS. 4, 6 and 7, the sample holder 22 comprises a washing cylinder 221, an external cylinder drive gear 222, and a lower frame 223. The lower frame 223 comprises two vertical side gear discs and a curved surface 2232 connecting the gear discs. The inward vertical surface of the vertical side gear disc is smooth. The cylinder driven gear 2231 on the outside part of the vertical side gear disc can mesh with the external cylinder drive gear 222. In the center of the vertical side gear disc is an orifice 2234 to enable a cylinder block 212 and a liquid outlet 214 to insert into. There is a curved surface 2232 underneath the washing cylinder 221. The curved surface 2232 connects with the vertical side gear discs, thereby form a washing/incubating space together with the two vertical side gear discs for the washing cylinder to rotate. Other than the sides connected with the vertical side gear disc, two other sides of the curved surface 2232 comprise a first lower frame pouring lip 2233 and a second lower frame pouring lip 2237.

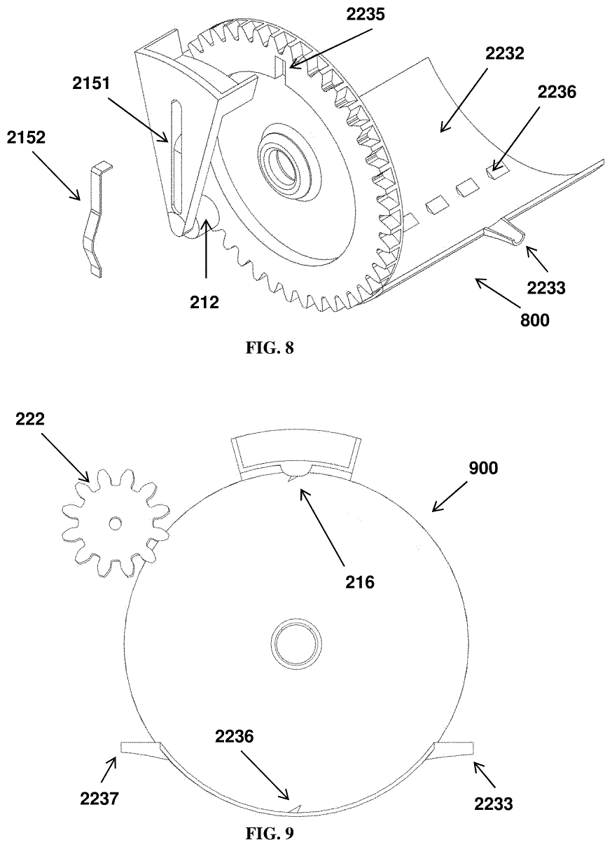

As shown in FIG. 8, the outer wall of the V-shaped block 215 can comprise a retaining slot 2151. The retaining slot 2151 engages with a retaining spring clip 2152. The lower portion of the retaining spring clip 2152 comprises a convex shape while the upper portion of the retaining spring clip 2152 comprises an inverted L shape. Further, the upper end of the retaining spring clip 2152 can snap into the groove 2235 on the vertical side gear disc of the V-shaped block 215. Therefore, when the retaining spring clip 2152 is snapped into the groove 2235, it can lock the position of the lower frame 223 of the sample holder. However, when the sample holder assembly 2 is inserted into support frame 4 of the device for incubating or washing a biological sample, the lower portion convex shape of the retaining spring clip 2152 can be pressurized, thereby push out the upper inverted L shape end of the retaining spring clip 2152 from the groove 2235. After the retaining spring clip 2152 is pushed out of the groove 2235, the lower frame 223 of the sample holder can move (i.e., swing or rotate) without restrictions.

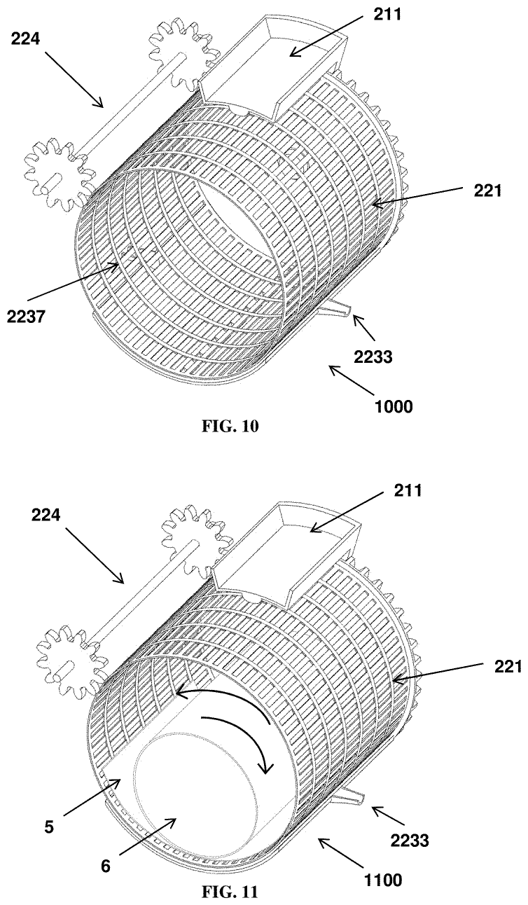

As shown in FIGS. 9 and 10, the washing cylinder 221 is a cylindrical structure formed by rolling a thin film sheet. A volume occupier 6, which is in a cylinder shape and is smaller than the washing cylinder 221, can be inserted inside the void of the washing cylinder 221. The volume occupier 6 can take space and force the liquid inside the sample holder 22 to spread over the Western blotting membrane 5, which resides between the inner surface of the washing cylinder 221 and the outer surface of the volume occupier 6. The washing cylinder 221 comprises a plurality of slots/openings on the thin film sheet to allow the free flow of liquid into and out of the washing cylinder. Further, the plurality of slots and openings can be poked or pushed by the first poking teeth 216 located on the bottom of the washing cylinder 221, and by the second poking teeth 2236 located on surface of the curved surface 2232. The first poking teeth 216 and the second poking teeth 2236 are configured to have the same orientation (clockwise or anticlockwise) with regard to the resulting movement of the washing cylinder when either of the first poking teeth 216 or the second poking teeth 2236 pokes the washing cylinder through the plurality of slots/openings on the thin film. Accordingly, when the sample holder 22 swings under the external force provided by the cylinder drive gear 222, the washing cylinder 221 swings/rotates as well; then the first poking teeth 216 and the second poking teeth 2236 poke the washing cylinder 221 and rotate the washing cylinder one way or the other.

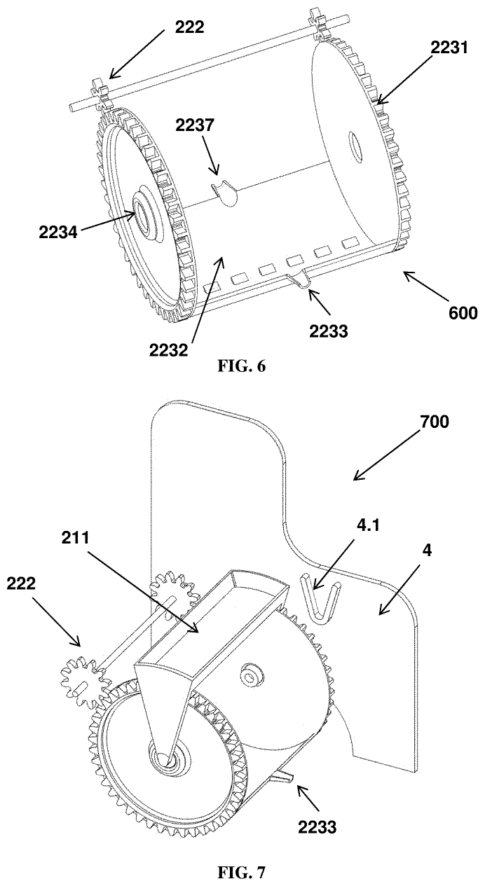

In some embodiments, as shown in FIGS. 6-11, when the upper basin 21 is fixed and the lower frame 223 of the sample holder 22 is driven by the external cylinder drive gear 222, the central orifice 2234 on the vertical side gear disc can be used as a rotation axis, the sample holder 22 together with the washing cylinder 221 can be swung back and forth depending on the movement of the external cylinder drive gear 222. At the same time of the swing, the washing cylinder 221 can be poked repeatedly by the first poking teeth 216 and the second poking teeth 2236 through the slots/openings on the thin film of the washing cylinder 221 such that the washing cylinder 221 can rotate inside the sample holder 22.

Alternatively the sample holder 22 can be moved by the sample holder drive gear 224 shown in FIGS. 9-11

As to the biological sample to be analyzed: The Western blotting membrane 5 to be incubated and washed is placed into the washing cylinder 221 and sometime adhered to the inner surface of the washing cylinder 221. The volume occupier 6 is also placed inside the washing cylinder 221 and press on the Western blotting membrane 5. When there is liquid inside the sample holder and over the curved surface 2232, the liquid tends to accumulate at the bottom. Without the volume occupier, the Western blotting membrane 5 may only be partially covered by the liquid. With the volume occupier inside and squeeze and spread out the liquid, more surface area of the Western blotting membrane 5 then without the volume occupier 6. When the first poking teeth 216 and the second poking teeth 2236 through the slots/openings on the thin film of the washing cylinder 221, the Western blotting membrane 5 rotates in circles together with the washing cylinder 221, thereby allowing the Western blotting membrane 5 to pass through the liquid at the bottom of the washing cylinder 221 and washing/incubating the Western blotting membrane 5 more thoroughly in smaller volume of the liquid when compared in the absence of the volume occupier 5 or the poking teeth 216/2236. In addition, the second poking teeth 2236 can push the Western blotting membrane 5 away from the inner surface of the washing cylinder 221 so that both surfaces of the Western blotting membrane 5 can be incubated/washed more thoroughly.

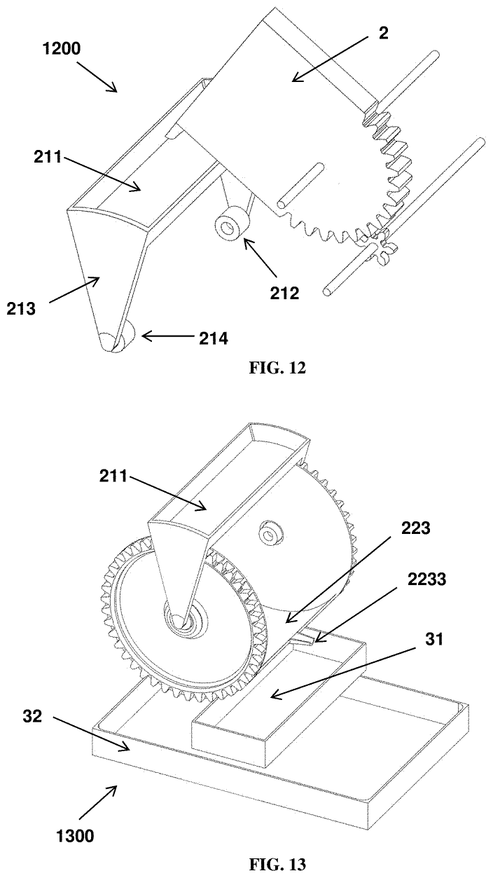

The movement of the liquid during the process can be shown in FIGS. 12-14. In some embodiments, the disclosure provides a method to incubate and wash the biological sample membrane or gel as follows. For example, the biological sample can be pre-treated; various solvents for the incubation and washing can be prepared or ordered; then the liquids can be placed and stored in the chamber 11 of the rotatable liquid storage container assembly 1. The rotatable liquid storage container assembly 1 can be driven by an external drive gear, thereby the chamber 11 can be rotated to tilt its body to such a degree that the liquid stored in the chamber 11 can pour out from the pouring lip 15, partly or completely, in the liquid collection basin 211 of the sample holder assembly 2, passes through the liquid conduit 213 on the vertical V-shaped stand. Then the liquid in the liquid conduit 213 can flow out the liquid outlet at the end of the liquid conduit 213 and into the sample 22. The lower frame 223 of the sample holder 22 can swings back and forth driven by the external cylinder drive gear 222, such that the liquid thoroughly contacts the biological sample at the lower frame 223 to complete the incubation and washing process.

According to FIG. 13, after the incubation and washing process is completed, the sample holder 22 can be controlled by an external motor to rotate clockwise or counterclockwise. AS shown in FIG. 13, when the sample holder 22 is rotated clockwise, the lower frame will tilt to such an angle that the liquid stored over the curved surface 2233 can be poured out through the first lower frame pouring lip 2233 into the antibody collection and recovery tank 31. The antibody collection and recovery tank 31 can be used when the first antibody is used in the Western blotting process. The antibody can be recovered. Then the sample holder 22 can be rotated back to the original position for the next washing/incubation step. An additional liquid can be added to the sample holder 22, and the next step can be performed. When the next washing/incubating step is completed, according to FIGS. 6, 9, 13 and 14, when the sample holder 22 is rotated counterclockwise, the lower frame will tilt to such an angle that the additional liquid stored over the curved surface 2233 can be poured out through the second lower frame pouring lip 2237 into the waste collection tank 32 placed within the liquid collection and recovery tank 3. The above steps can be repeated until the whole washing/incubating steps are performed.

The biological sample can be kept in moisture and stayed inside the sample holder and in contact with certain liquid, even in a constant swinging motion when controlled by an external motor, until the user retrieves the biological sample.

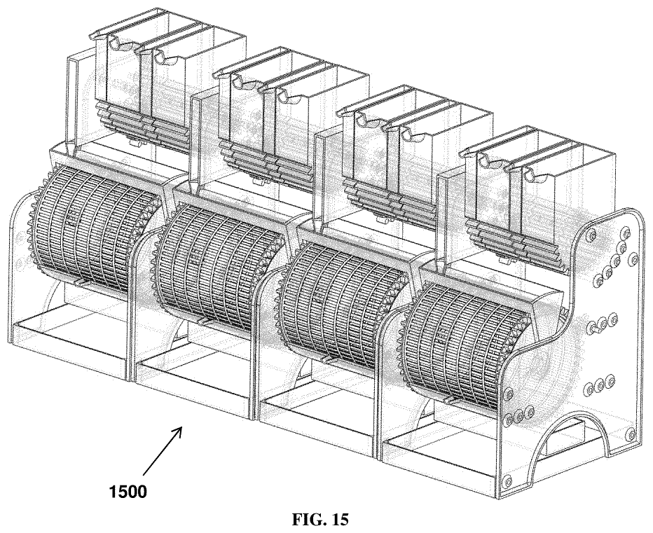

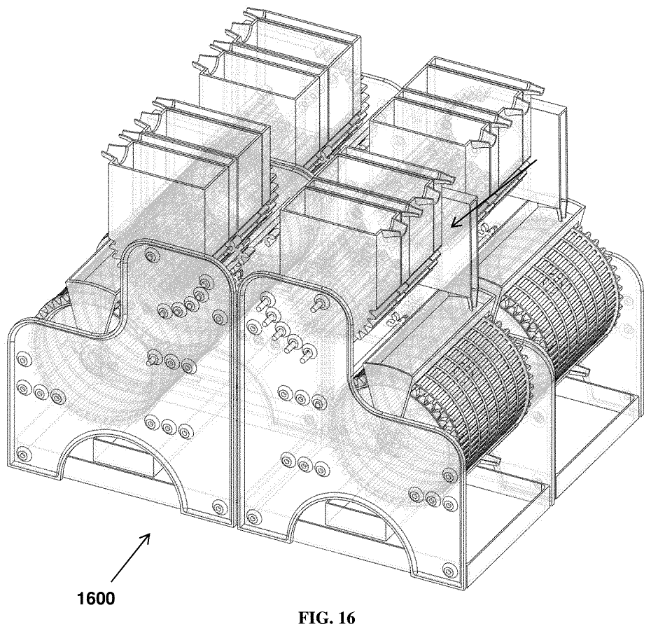

As shown in FIGS. 15 and 16, the device for incubation and washing of biological samples can be operated using a single device format, or in a multi-device formats, such as those shown in FIGS. 15 and 16, in series or in tandem.

As described above, the device of the present disclosure can accomplish the followings:
1) Use less liquid and thoroughly wash/incubate the Western blotting membrane. This can be accomplished by the presence of the volume occupier and the constant poking and rotating the Western blotting membrane during the process. The reduction in the amount of antibody used can lead to the reduction of costs when conducting the Western blotting experiment.
2) By keeping the upper basin 21 in a fixed position and allowing the lower frame 223 movable under the control of an external drive gear, sample holder can be rotated under controlled conditions. By moving the sample holder in circles using the poking teeth, the washing cylinder and the Western blotting membrane inside thereof can be rotated in a circle, which in turn drives the biological samples through the liquid stored over the surface of the curved surface 2232 for better washing/incubating/contact.
3) The poking teeth on the curved surface help pushing the Western blotting membrane upwards and away from the inner surface of the washing cylinder. Therefore, the poking motions help agitate the liquid and force the member to move upwards and downwards in the liquid to achieve better and more thorough washing of both sides of the Western blotting membrane.
4) The rotation angle of the lower frame 223 can be controlled. When the angle is small, the first lower frame pouring lip 2233 and the second lower frame pouring lip 2237 can be kept higher than the level of the liquid over the curved surface 2232 such that the liquid will be within the sample holder 22. When the angle is big enough, the first lower frame pouring lip 2233 or the second lower frame pouring lip 2237 can become lower than the level of the liquid over the curved surface 2232 such that the liquid will be poured out of the sample holder.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

We claim:

1. A device for incubating or washing a biological sample, the device comprising:
(a) a support frame comprising an upper part and a lower part;
(b) a rotatable liquid storage container assembly disposed at the upper part of the support frame, wherein the rotatable liquid storage container is configured to store and transfer a liquid; and
(c) a sample holder assembly disposed at the lower part of the support frame, wherein the sample holder comprises:
(i) a sample holder configured to receive a biological sample; and
(ii) an upper basin disposed between the sample holder and the rotatable liquid storage container; wherein the upper basin is engaged with both the sample holder and the rotatable liquid storage container assembly.

2. The device of claim 1, wherein the rotatable liquid storage container assembly comprises a first liquid storage container and a first drive gear, wherein the first liquid storage container comprises a first chamber comprising (i) an open-ended top, (ii) two vertical sides opposing each other, and (iii) a bottom side comprising matching driven gear to mesh with the first drive gear, thereby the first drive gear drives the first liquid storage container.

3. The device of claim 2, wherein each of the two vertical sides of the first chamber comprises an opening configured for a rod to pass through, wherein the first liquid storage container further comprises an inserted container disposed in the first chamber, wherein the inserted container comprises a cavity configured for the rod to pass through, and wherein the rod secures the first liquid storage container to the support frame.

4. The device of claim 2, wherein the first chamber further comprises a pouring lip on the open-ended top.

5. The device of claim 2, wherein the rotatable liquid storage container assembly further comprises a second liquid storage container and a second drive gear, wherein the second liquid storage container comprises a second chamber, wherein the second drive gear drives the second liquid storage container.

6. The device of claim 5, wherein the first drive gear is configured not to drive the second liquid storage container, and wherein the second drive gear is configured not to drive the first liquid storage container.

7. The device of claim 5, wherein the first drive gear and the second drive gear are configured not to share a common coordinate on any of the X-axis, Y-axis, and Z-axis of a 3-dimensoional Cartesian coordinate system.

8. The device of claim 1, further comprising (d) a liquid collection and recovery tank disposed at the lower part of the support frame and below the sample holder assembly.

9. The device of claim 1, further comprising a volume occupier disposed within the sample holder.

10. The device of claim 1, further comprising a sample holder drive gear configured to operate the sample holder.

11. The device of claim 1, wherein the sample holder further comprises: a washing cylinder disposed within the sample holder, wherein the washing cylinder is cylindrical with two open ends, and wherein the washing cylinder comprises a plurality of slots.

12. The device of claim 11, wherein the sample holder further comprises: a curved surface at the lower part of the sample holder; wherein the curved surface comprises a plurality of poking teeth configured to poke the washing cylinder by protruding through the plurality of slots.

10. A system comprising a plurality of the device according to claim 1.

11. The system of claim 9, wherein the system further comprises a processor to control the plurality of the device.

12. The system of claim 10, wherein the processor is configured to control the operation of rotatable liquid storage container assembly and the sample holder.
